(19) United States
(12) Patent Application Publication
(10) Pub. No.: US 2020/0060546 A1
(43) Pub. Date: Feb. 27, 2020

(54) A System and Method for Monitoring Human Performance

(71) Applicant: Akash Madnani, Nagpur (IN)

(72) Inventors: Akash Madnani, Nagpur (IN); Vivek Sharma, Nagpur (IN); Javed Sheikh, Nagpur (IN); Pratap Shukla, Nagpur (IN); Manish Chandra, Navi Mumbai (IN); Aelish Azad, Nagpur (IN); Swapnil Meshram, Allapalli (IN); Umesh Hassani, Nagpur (IN); Anaadi Shivnani, Nagpur (IN); Anil Paghadal, Nagpur (IN)

(21) Appl. No.: 16/609,535

(22) PCT Filed: May 2, 2018
(86) PCT No.: PCT/IB2018/053023

§ 371 Date: Oct. 30, 2019

(30) Foreign Application Priority Data
May 8, 2017 (IN) 201721016237

(51) Int. Cl. A61B 5/00; A61B 5/11; A61B 5/0205

(52) U.S. Cl. CPC A61B 5/0022; A61B 2562/0219; A61B 5/0205; A61B 5/1118

(57) ABSTRACT

The present invention is to provide a system and a method for monitoring human performance. The system is provided with a wearable device and an electronic device. The wearable device can be worn on a torso. The wearable device having a sensing system configured to sense parameters such as breathing pattern, activity, posture, sleeping pattern, ambience. Further sensed data is wirelessly sent to a second processing unit of the electronic device from a first processing unit of the wearable device for processing the data. And comparing the data with a calibrated and fed data to calculate a mental wellness and physical wellness of a user or group. The data is stored on a cloud storage, where a third application module configured to process the historic data with the mental wellness and physical wellness and for providing suggestions to enhance the mental wellness and physical wellness.

### FIELD OF THE INVENTION

The present invention relates to a system and a method for monitoring human performance. More particularly the present invention relates to a system and a method for monitoring human performance such as breathing pattern, activity, posture, sleeping pattern, ambience, work performance, health and the like to improve mental wellness and physical wellness.

### ABBREVIATIONS USED

FSR—Force Sensing Resistor

PCB—Printed Circuit Board

GPS—Global Positioning System

BMI—Body Mass Index

BPM—Breath per Minute

### BACKGROUND OF THE INVENTION

Presently, there has been an increased rate of people who are suffering from stress and the related sufferings. There are many hidden and small factors that create stress in people. Some factors include breathing, quality of sleep, activity, posture, ambience that affects an individual's health and performance. In a recent survey, stress has ranked as a major risk factor globally. Due to stress, people may be affected by diseases and their work efficiency, performance, focus, may get reduced eventually. Also, it has been observed that almost 95% of corporate employees are under stress and due to which their performance is hampered. Even students are also affected by stress due to their vast study and exams.

In recent research, it has come to notice that in eight working hours, the productivity of an average employee is only approximately three hours. As the productivity of the employee is proportional to the value of the organization, it adversely affects the organization as well. Employers are trying to implement various systems and methods such as time management systems, personnel management systems, accounting systems and the like to maintain and track resources within the organization to boost the performance of the employees. But tracking of employee's performance statistics and physical presence is not the solution to the problems. By knowing the employees mental and physical situation and to overcome the same is more helpful to improve the efficiency.

US 20016742 A1 discloses a process for measurement and monitoring the stress level by processing heart rate variance data collected during a stress test, wherein the heart rate variance data may include a coherence attribute, and transmitting the heart rate variance data over a network and to a specified location which delivers numeric score to the end user. Thus, the method attempts to predict and monitor human stress solely based on the heart rate which can neither be helpful in predicting slight variations in the stress levels nor helps the user for assessment of alertness level of the user.

U.S. Pat. No. 5,433,223 to M. Moore-Ede et al. describes a method for predicting the likely alertness level of an individual at a specific point in time based upon a mathematical computation of a variety of factors that bear some relationship to alterations in alertness. The individual's Baseline Alertness Curve (BAC) is first determined based on five inputs and represents the optimal alertness curve displayed in a stable environment. Next, the BAC is modified by alertness modifying stimuli to arrive at a Modified Baseline Alertness Curve. Thus, the method is a means for predicting an individual's alertness level, not cognitive performance.

Therefore, there is a need of a system and a method to track people's day to day routine and collect real-time data behind their stress and reason for the lack in performance and overcome few or more problems mentioned above.

### OBJECTS OF THE INVENTION

An object of the present invention is to provide a system and a method for monitoring human performance.

One more object of the present invention is to provide a system and a method for monitoring human performance, wherein the system keeps real-time track of mental wellness and physical wellness of a user.

Another object of the present invention is to provide a system and a method for monitoring human performance, wherein the system identifies a problem behind not having the mental wellness and the physical wellness for the user in particular period of time.

Yet another object of the present invention is to provide a system and a method for monitoring human performance, wherein the system can send an alert to the user when their evaluated parameters are not in correlation with the identified fed and calibrated parameters.

Another one more object of the present invention is to provide a system and a method for monitoring human performance, wherein the system helps the user to check breathing pattern, activity, posture, sleeping pattern and ambience.

Further object of the present invention is to provide a system and a method for monitoring human performance, wherein the device can process and provide data to an individual as well as to a corporate company for enhancement of work efficiency of the individual as well as group.

Further one object of the present invention is to provide a system and a method for monitoring human performance, wherein work efficiency of the user may be collected through a wearable device and further processed for assessment by human resource team of an organization to initiate required corrective actions.

Further one more object of the present invention is to provide a system and a method for monitoring human performance, wherein the system is capable to assist and monitor the user in real time.

Still one object of the present invention is to provide a system and a method for monitoring human performance, which can analyze the user's personality as well as their compatibility with others.

### SUMMARY OF THE INVENTION

According to the present invention, there is provided a system for monitoring human performance. The system comprising a wearable device, an electronic device, and a cloud storage.

The wearable device is having a sensing system configured to sense parameters such as breathing pattern, activity, posture, sleeping pattern, and ambience and sends data to a first processing unit having a first processing module. The wearable device is worn on a torso of a user.

The electronic device communicates wirelessly with the wearable device. The electronic device having a second processing unit configured to receive the data from the first processing module. The second processing unit processes the data and sends data to a second processing module of the electronic device and compares with a calibrated and fed data to calculate mental wellness and physical wellness. It is notified to the user in real time through the second processing module. The data and the calculated state of the mental wellness and the physical wellness are stored on a cloud storage periodically, the breathing pattern, sleeping pattern, posture, activity, and ambience data is also stored on the cloud storage.

A third application module configured in the cloud storage, the third application module process the historical data of the mental wellness and the physical wellness of the user and the data of other users to identify patterns and accordingly providing suggestions to improve the mental wellness and the physical wellness.

The sensing system comprises a light sensor, a noise sensor, a temperature sensor, an accelerometer, a gyroscope, a magnetometer, a FSR sensor and a strain gauge. The breathing pattern is sensed by the FSR, the strain gauge, and the accelerometer. The second processing module analyses breathing patterns of the user to define a mental state. The activity of the user is sensed by accelerometer, gyroscope, and magnetometer and the processed data is used to evaluate the physical wellness and mental wellness of the user. The posture is calibrated by reading standing straight posture and sitting straight posture. The posture is sensed by the accelerometer, the gyroscope, the magnetometer and the strain gauge. Through GPS of the electronic device geographical location of the user corresponding to the data is stored for evaluating location wise performance and mental wellness and physical wellness of the user.

The wearable device is set in night mode before sleeping; during night mode the temperature, light and noise data is collected, and the data is sent to the first processing unit. The ambience is sensed by the light sensor, the noise sensor, and the temperature sensor.

User groups can be created in the cloud, and the aggregate data can be displayed on the electronic device (such as mobile, computer, tablet and the like) through the second processing module.

In another aspect of the present invention, there is to provide a method for monitoring human performance. The method comprising a step of sensing the parameter such as breathing patterns, activities, postures, sleeping patterns, and ambience by a sensing system.

Thereafter, sending user's data from the sensing system to a first processing module in of a wearable device.

Further, sending the user's data from the first processing module to a second processing module on an electronic device.

Thereafter, analyzing and comparing the received data with a calibrated and fed data such as name, gender, age, weight and the like, to evaluate performance and a mental wellness and physical wellness of the user by the second processing module in the electronic device and is notified to the user in real time through the second processing module.

Further, storing the evaluated performance and the mental wellness and physical wellness data in a cloud storage.

Processing the evaluated historical performance and the mental wellness and physical wellness data and the data of other users to identify patterns and accordingly providing suggestions to the mental wellness and physical wellness to improve the user's lifestyle.

Geographical location of the user corresponding to the data can be stored for evaluating location wise performance and the mental wellness and physical wellness of the user.

### BRIEF DESCRIPTION OF THE DRAWINGS

The advantages and features of the present invention will become better understood with reference to the following detailed description and claims taken in conjunction with the accompanying drawings, wherein like elements are identified with like symbols.

FIG. 1 shows a block diagram of a system for monitoring human performance in accordance with the present invention;

FIG. 2 illustrates a back-perspective view of a wearable device as shown in FIG. 1;

FIG. 3 illustrates a front-perspective view of the wearable device as shown in FIG. 1;

FIG. 4 illustrates the wearable device with a provision to be arranged on the users clothing or on the pillow in accordance with the present invention;

FIG. 5 illustrates a sectional view of the wearable device;

FIG. 6 illustrates the arrangement of the wearable device in a vibrating mode;

FIGS. 7a, 7b, and 7c illustrate various positions at which the wearable device can be arranged on a user or near the user while sleeping;

FIGS. 8a, 8b, and 8c illustrate various postures measured by the wearable device for calibration;

FIG. 9 shows a schematic representation of the system in accordance with the present invention;

FIG. 10 illustrates the mental state of the user defined by a waveform of breathing pattern;

FIG. 11 illustrates a flowchart of working of an FSR and a strain gauge and accelerometer for sensing breathing pattern of the user;

FIG. 12 illustrates a working flowchart of identifying posture and activity of the user;

FIG. 13 illustrates a working flowchart for identifying sleep and ambience around the user in the night;

FIG. 14 illustrates a working flowchart for identifying breathing pattern of the user;

FIGS. 15a, 15b, and 15c shows pictorial example of the working of the system; and

FIG. 16 illustrates a method for monitoring human performance in accordance with the present invention.

### DETAILED DESCRIPTION OF THE INVENTION

An embodiment of this invention, illustrating its features, will now be described in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

The present invention is to provide a system and a method for monitoring human performance. The system has a wearable device, which keeps real-time track of a mental and a physical wellness of a user. Specifically, the system analyzes a reason behind stress for the user in particular period of time. Further, the system can send an alert to the user when these parameters are not matched with a fed or calibrated parameter. The system helps the user to check breathing pattern, activity, posture, sleeping pattern and ambience. The system can process and provide data to an individual as well as to a corporate company for enhancement of work efficiency of the individual as well as group. The work efficiency of the user is calculated through the processing of the collected data for assessment by human resource team of an organization to initiate required corrective actions. Further, the system is capable to assist and monitor the user in real time. Also, the system can analyze the user's personality as well as their compatibility with others.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced items.

The disclosed embodiments are merely exemplary of the invention, which may be embodied in various forms.

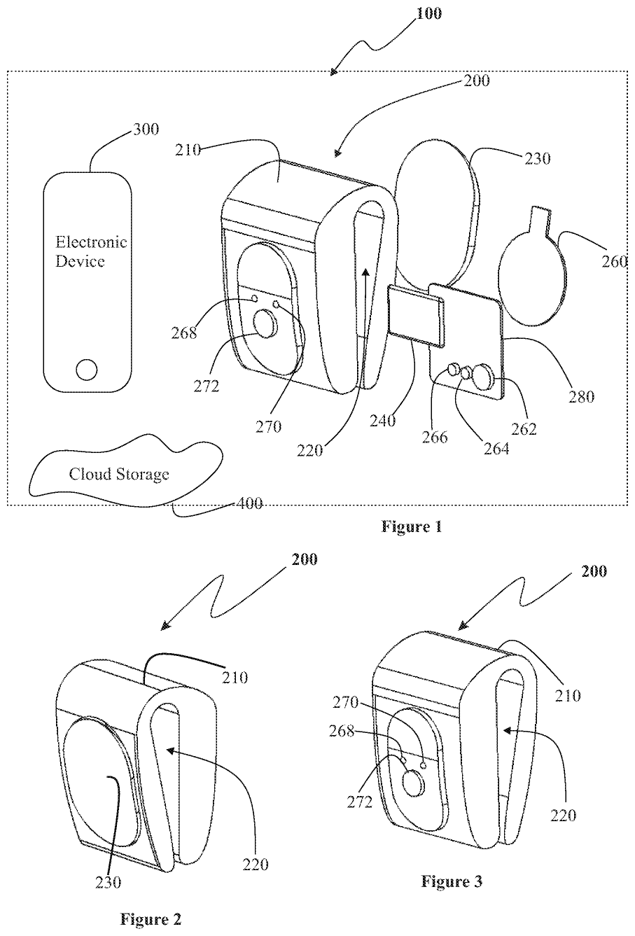

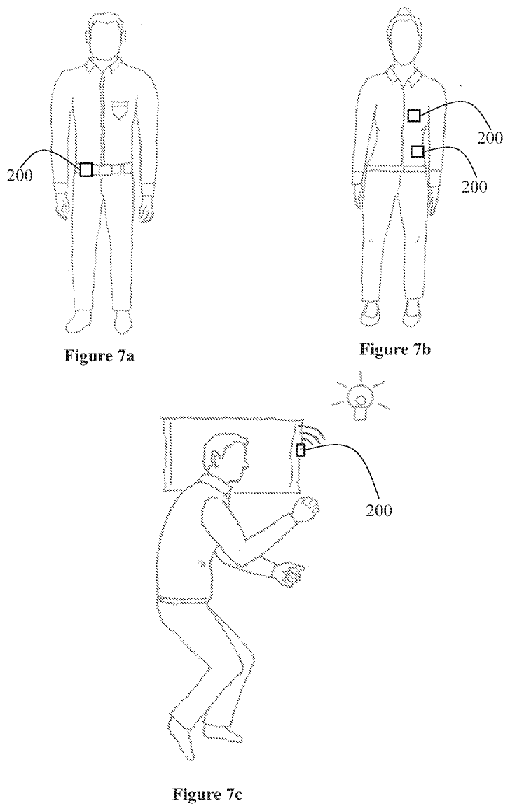

Referring now to FIG. 1, a system 100 for monitoring human performance in accordance with the present invention is illustrated. The system 100 includes a wearable device 200, an electronic device 300 and a cloud storage 400. The wearable device 200 can be worn anywhere on a torso of a user. The wearable device 200 can be secured over inner-wears of a user. Specifically, a male user can wear the wearable device 200 near their waist as shown in FIG. 7a and a female user can wear the device on their clothing at the waist or on the torso near their breast portion on a bra as shown in FIG. 7b according to her convenience. Also, the wearable device 200 needs to mount on a pillow on which the user is resting his/her head while sleeping as shown in FIG. 7c. It may be obvious to a person skilled in the art to wear or mount the wearable device 200 on any other portion of a torso.

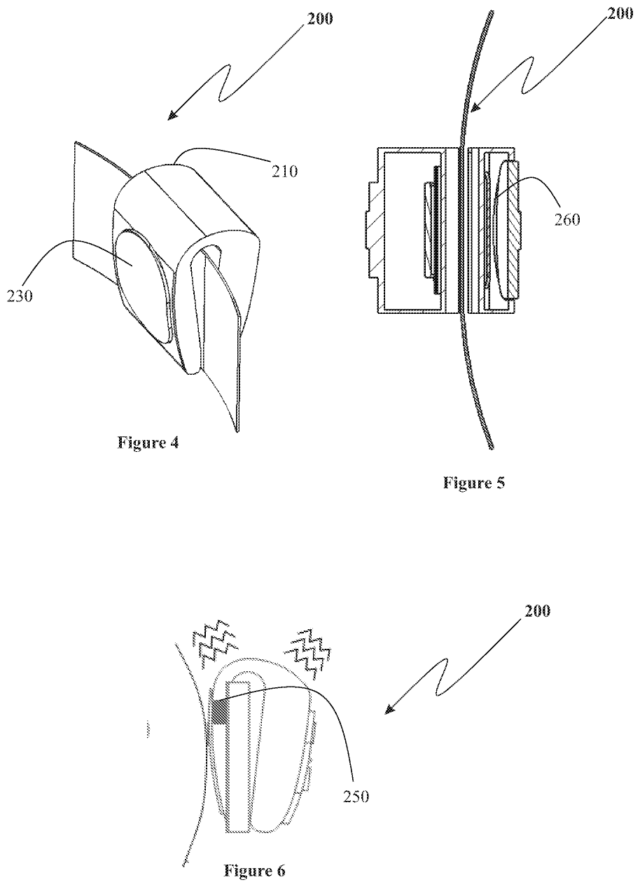

Referring now to FIGS. 2, 3 and 4 the wearable device 200 is provided with a casing 210. The casing 210 helps the wearable device 200 to protect from external impacts and therefore damages to the internal parts thereof. Further, the wearable device 200 is configured in a form of clip, which can be clipped to clothes of the user, specifically undergarments like, briefs, bra, panties or any other similar undergarment. This wearable device 200 has a cavity 220 for holding or hooking the wearable device 200 on the clothes of a user as shown in FIGS. 7a, 7b, and 7c. Specifically, the cavity 220 is for hooking and holding the wearable device 200 on the clothes of the user in such a way that the wearable device 200 is capable of sensing with the body of the user for collecting inputs therefrom. Further, an actuator 230 is provided on the wearable device 200 for actuating the wearable device 200 for performing sensing and other related operations. Specifically, the actuator 230 is facing towards the user's body and attached tightly to sense the force or pressure applied by the user's body on the wearable device 200. A power source 240 such as a battery is placed inside the wearable device 200. The power source can be a removable and rechargeable power source 240. The power source 240 provides power to all the elements of the wearable device 200 for their functioning. FIGS. 4 and 5 shows a cloth clamped by the wearable device 200.

Further, the wearable device 200 is having a sensing system (not numbered) and a vibrating motor 250 (as shown in FIG. 6) and a first processing unit (not shown). The sensing system facilitates in sensing various parameters, such as a breathing pattern, an activity, a posture, a sleeping pattern of the user and an ambience around the user while sleeping. This improves the accuracy of determining the user's performance by indicating the factor which affects their performance. Specifically, the sensing system is having sensors, such as an FSR (force sensing resistor) sensor along with a strain gauge 260 and an accelerometer 262, a gyroscope 264, a magnetometer 266, a light sensor 268, a noise sensor 270, a temperature sensor 272. These sensors of the sensing unit are placed on a PCB 280 as shown in FIG. 1.

Further, the most important factor that determines mental wellness and physical wellness of the user is his/her breathing pattern. The breathing pattern is measured and categorized in various states such as an attentive, a composed, a stressed, a depressed state and the like. It may be obvious to a person skilled in the art to measure the breathing pattern in some other obvious states. In the present embodiment, the breathing pattern is sensed by using the FSR sensor and the strain gauge 260 and the accelerometer 262 as shown in FIGS. 2, and 5. These sensors are used together to increase data accuracy of the breathing pattern.

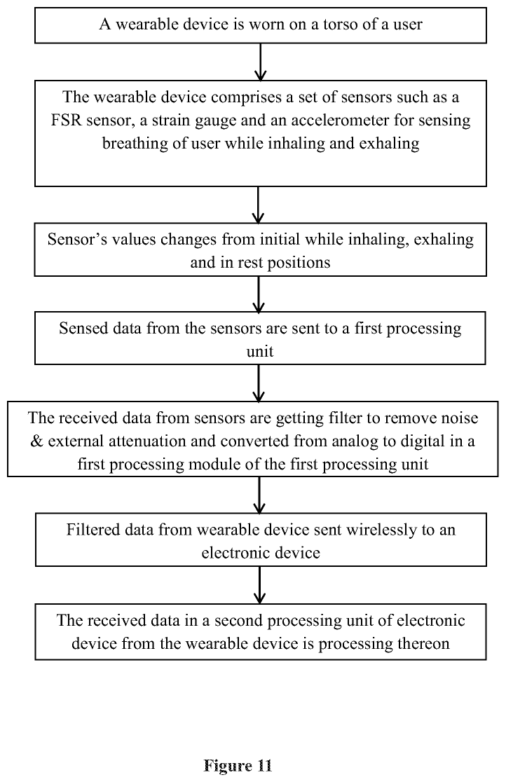

Specifically, FIG. 11 shows a flow of chart of working of the FSR, the strain gauge 260 and the accelerometer for sensing breathing pattern of the user. The wearable device 200 has to be worn on a torso of the user. Thereafter, the FSR, the strain gauge 260 and the accelerometer senses breathing of user by measuring the torso movement while inhaling and exhaling of the user by measuring the change in values of the FSR, the strain gauge 260 and accelerometer. The sensed data from the sensors are sent to the first processing unit on the wearable device 200. The received data from sensors are filtered to remove noise & external attenuation and converted from analog to digital in the first processing module of the first processing unit. The filtered data from wearable device sent wirelessly to the electronic device 300. The received data in the second processing unit of the electronic device 300 from the wearable device 200 is processed thereon.

Referring now to FIG. 14, a working flowchart for identifying breathing pattern of the user in accordance with the present invention is illustrated. The FSR, the strain gauge 260 and the accelerometer senses breathing of user by measuring the torso movement while inhaling and exhaling of the user by measuring the change in values of the FSR, the strain gauge 260 and accelerometer. Sensor's values change from initial when inhaling, exhaling and rest positions are sensed. Sensed data from the sensors are sent to the first processing unit for processing. Further, the processed data is received by the second processing unit of the electronic device 300 from the wearable device 200. The data is converted from hexadecimal to decimal in the second processing unit. The decimal data is filtered again in the second processing module for refining the waveforms of the breath pattern. Waveform of breath pattern is further passed through a mathematical model in the second processing unit for calibrating the data and to generate breath per minute (bpm) value. Continuous measuring, storing and correlating this data to a state of mind is done in the second processing unit. This data is compared with the calibrated data and the fed data in second processing unit. This data is also sent to a third processing module in the cloud storage 400 as shown in FIGS. 15a, 15b & 15c.

The FSR sensor is adapted to give a linear response to lower force ranges. If the sensor data is not proportional to the applied load, the strain gauge 260 manages to respond to higher force ranges. Also, the strain gauge 260 has a linear response for applied loads. Further, the strain gauge 260 is adapted to capture force and variations in the force applied to the strain gauge 260 more accurately than the FSR sensor. Thus, using a strain gauge 260 along with the FSR sensor leads to higher data accuracy in a larger load for better results.

Specifically, in the present embodiment, the FSR sensor along with the strain gauge 260 is used for sensing a pressure and a force applied thereon by movement of the user's body during inhaling and exhaling process or during bending. Also, the strain gauge 260 is adapted to sense the applied force, pressure on the wearable device 200 during inhale and exhale process to improve the accuracy of the breathing pattern. Further, the accelerometer 262 is configured thereon for detecting and monitoring the linear movements of the wearable device 200.

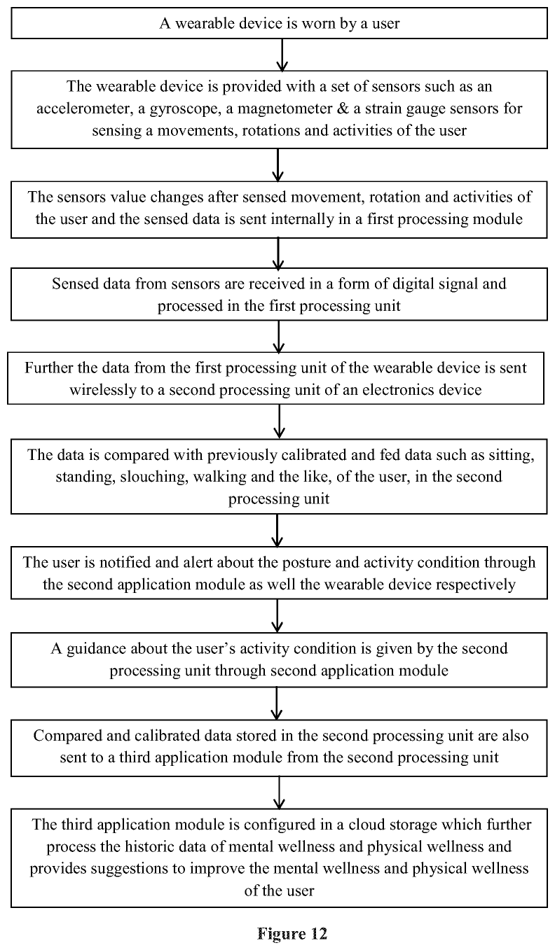

Further, in the present embodiment, the posture of the user is calibrated manually and fed to the system 100 as a reference data. For every user, the posture may be calibrated in a straight standing posture and a straight sitting posture. It may be obvious to a person skilled in the art to calibrated posture in other states. Specifically, the posture is sensed by the accelerometer 262, the gyroscope 264, the magnetometer 266 and the strain gauge 260. The calibrated data is set by the user before start using the wearable device 200. Specifically, FIG. 12 shows a working flowchart of identifying posture and activity of the user. The change in values of the accelerometer 262, the gyroscope 264, the magnetometer 266 and the strain gauge 260 after movement, rotation, and activities of the user is sent internally in the first processing module. Sensed data from sensors, such as the accelerometer 262, the gyroscope 264, the magnetometer 266 and the strain gauge 260 are taken in a form of digital signal and processed in the first processing unit. Further, the data from the first processing unit of the wearable device is sent wirelessly to the second processing unit of an electronic device.

Further, the data is compared with previously calibrated and fed data such as sitting, standing, slouching, walking and the like, of the user, in the second processing unit. The user is notified and alerted about the posture and activity condition through the second application module as well the wearable device respectively in real-time. The second processing unit gives a guidance about the user's activity condition through the second processing module. The data is compared with the calibrated data stored in the second processing unit and also sent to the third application module from the second processing unit. The third application module is configured in the cloud storage 400 which further process the historical data of mental wellness and physical wellness and provides suggestions to improve the mental wellness and physical wellness of the user.

Furthermore, in the present embodiment, the activity of the user is also monitored in the system 100 for determining the usage of energy or calorie by the user. The activity such as walking, standing, running and the like are monitored using the accelerometer 262, the magnetometer 266 and the gyroscope 264. Also, the sleeping pattern of the user is monitored for finding the quality of sleep the user gets on a daily basis.

The sensed data of the sensing system 100 is further sent to a first processing unit. The first processing unit is configured on the wearable device 200. The first processing unit facilitates to process the sensed data received from the sensing system of the wearable device 200 using a first processing module (not shown). Specifically, the first processing unit removes noise and external attenuation from the received data from the sensing system. The FSR sensor, the strain gauge 260 and accelerometer 262 further converts the pressure and movement signal received from the actuator to an electrical signal. This electrical signal is filtered using combination of RC filters for noise reduction and then given to the analog to digital converter of the microprocessor of the first processing unit. And further, converting the filtered data from analog to digital and sending it to the first processing module of the wearable device 200. Internal signal processing is done in the microprocessor and transferred to the internal communication protocol for transferring data to the algorithm of a second processing unit in the electronic device. Furthermore, the processed data from the first processing unit of the wearable device 200 is sent to the second processing unit of the electronic device 300 in real time. The electronic device 300 can be a mobile phone or a smartphone or a computer or a tablet or any other similar devices which can synchronize the data from the wearable device 200.

The electronic device 300 is also having a second processing module for analyzing and displaying the data from the device. In the present embodiment, the second processing module is a software application which is operably configured inside the electronic device 300. Specifically, the wearable device 200 is communicating wirelessly via Bluetooth or near field communication or Wi-Fi communication protocol with the electronic device 300.

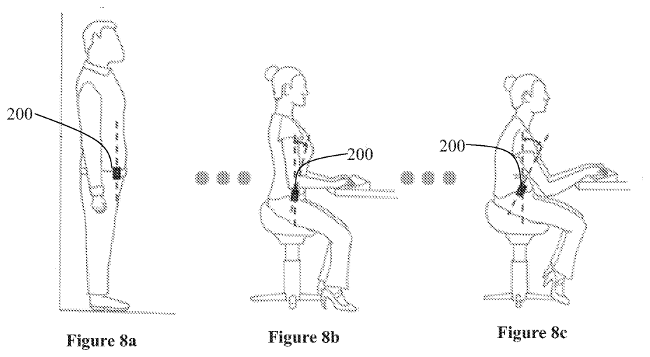

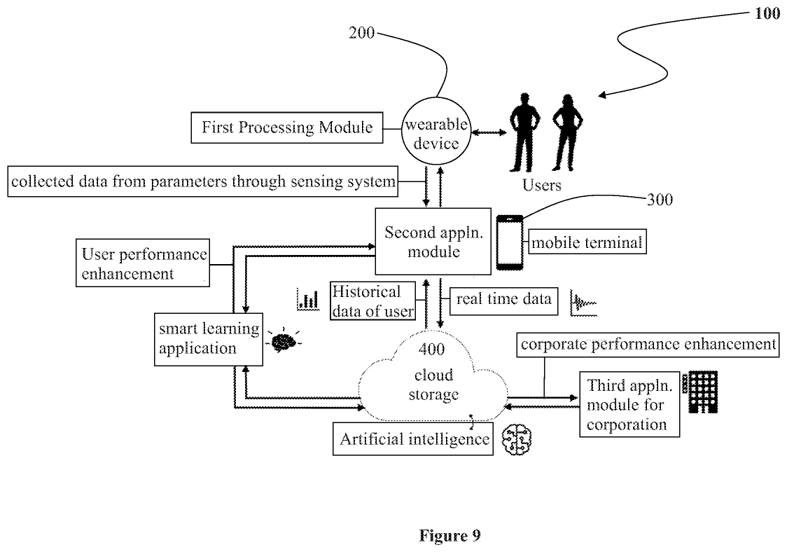

Further, the received data from the first processing unit is processed and compares with the calibrated and fed data in the second processing unit to calculate mental wellness and physical wellness and accordingly notifications are sent to the user in real time through the second processing module. The user has to feed the system 100 with the data such as name, age, gender, height, weight and different postures such as the posture is calibrated in various states such as slouching, standing straight and sitting straight and the like as shown in FIGS. 8a, 8b, and 8c. It may be obvious to a person skilled in the art to provide other calibrated data in various postures of the user which may bring an effect to user's lifestyle for precise calculation of the stress factor.

Further, the processed and calculated data from the electronic device 300 is stored on the cloud storage 400 periodically. Specifically, the breathing pattern, posture, activity, sleeping pattern and ambience data stored on the cloud storage 400. Further, a third application module is configured in the cloud storage 400. The third application module process the historical data of parameters such as the breathing pattern, sleeping pattern, posture, activity, and ambience data and the like to identify the mental wellness and the physical wellness of the user. And also, the data of other users to identify patterns and accordingly providing suggestions to improve the mental wellness and the physical wellness to improve the user's lifestyle. The third application module establishes two-way communication with the second processing module. The communication is established for sending the output data from the second processing module to the third application module. The third application module compares and analyzes the output data from the second processing module and sends it back to the second processing module which can be displayed on the electronic device 300. The third application module process the historical data of stress and can provide data for providing suggestions to reduce stress by a smart learning application. The electronic device 300 and the cloud storage 400 can communicate through internet.

The third application module is also capable of receiving and aggregating output data from various users wearing the wearable device 200, which can be displayed on the electronic device 300 or any such terminals. Also, the third application module is having artificial intelligence to compare and analyze the stress factor and is capable of determining the mental state of the user. Further, fine-tuning of the data is done in the third processing unit on the cloud storage 400. Long-term reports of the users are generated.

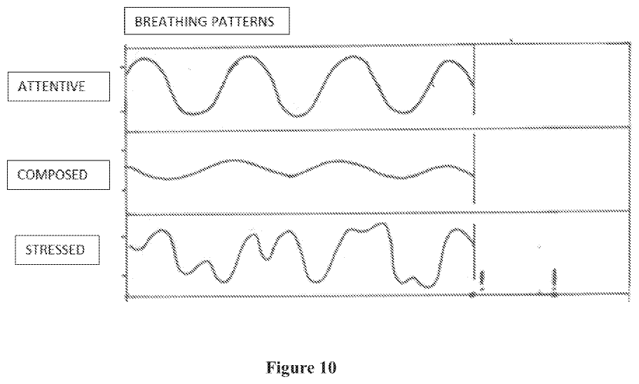

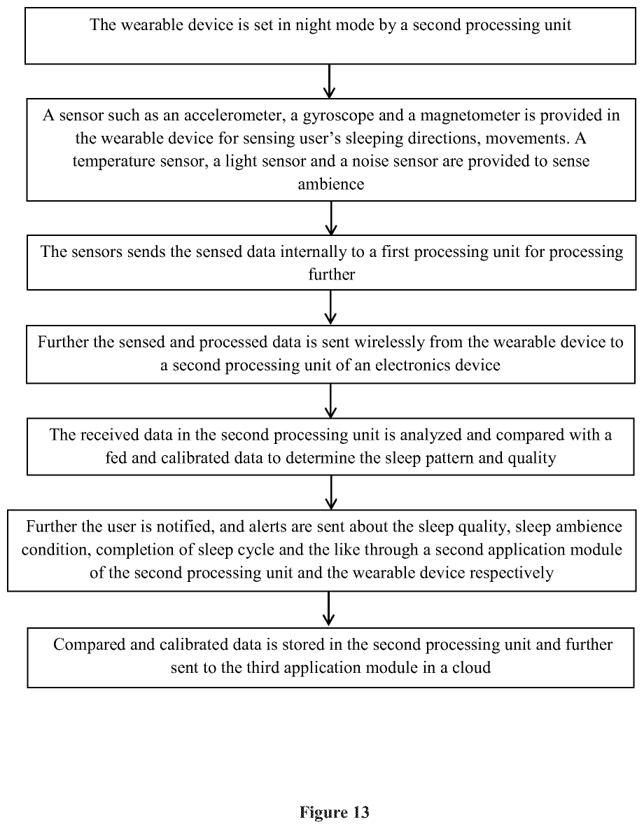

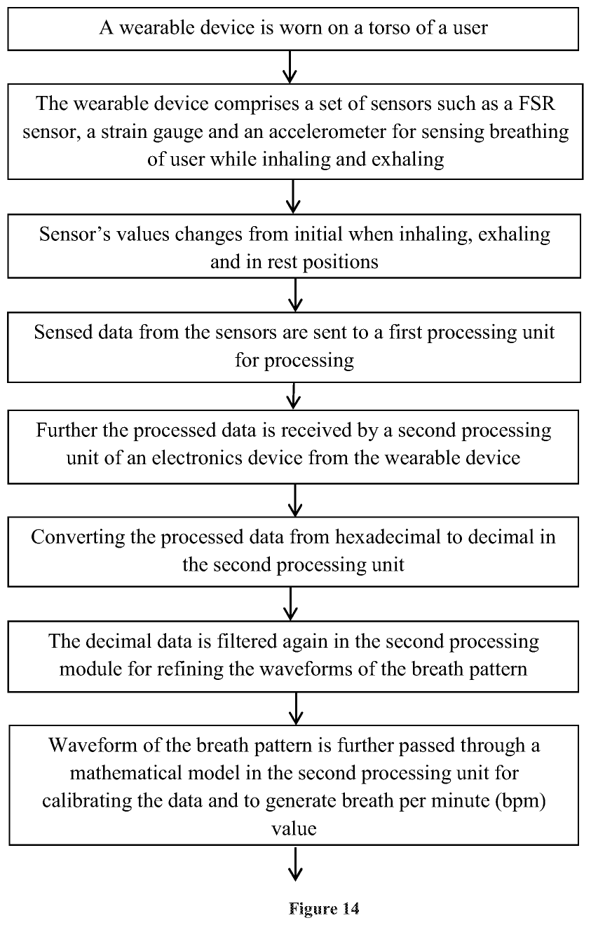

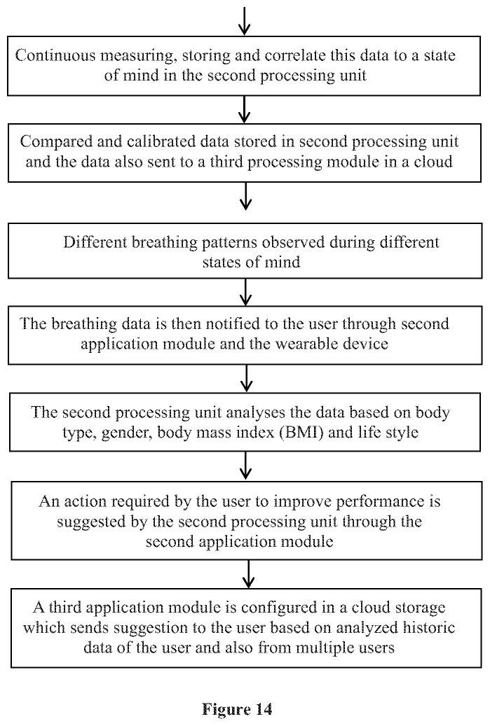

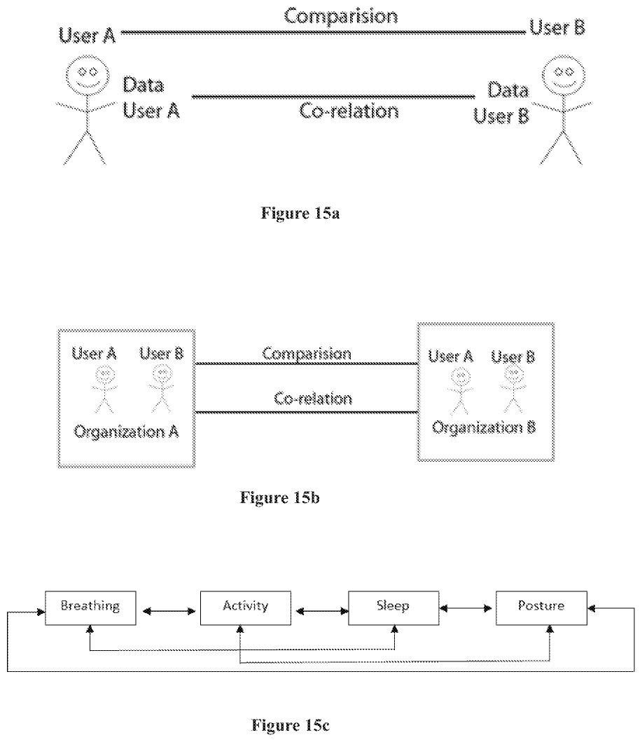

Further, the mental state of the user is defined by a waveform of breathing pattern as shown in FIG. 10. The wearable device 200 is switched to night mode before sleeping. During night mode the ambience such as light, noise, and temperature is sensed by using the light sensor, the noise sensor, and the temperature sensor respectively. Further, the data is sent to the processing unit, which further processes and analyses the data and communicate with the first processing module while synchronization with the device. Specifically, FIG. 13 shows working flowchart for identifying sleep and ambience around the user in the night. The sensors such as the accelerometer, the gyroscope, and the magnetometer are provided in the wearable device for sensing user's sleeping directions, movements and a temperature sensor, a light sensor, and a noise sensor are provided to sense ambience. The sensors send the sensed data internally to the first processing unit for processing further. The sensed and processed, this data is sent wirelessly from the wearable device 200 to the second processing unit of the electronic device 300. The received data in the second processing unit is analyzed and compared with a fed and calibrated data to determine the sleep pattern and quality.

Further, the user is notified and alert about the sleep quality and sleep ambience condition, completion of sleep cycle and the like through a second application module of the processing unit and the wearable device respectively. Compared and calibrated data is stored in the second processing unit and further sent to the third application module in the cloud storage 400. Real-time action required by the user to perform is suggested by the second processing unit via the second application module while the third application module suggests actions based on multiple data points from wider data sets of the user and other such users.

Different postures of the user such as postures while sitting, standing, etc. as shown in FIGS. 8a, 8b and 8c is sensed by the accelerometer 262, the gyroscope 264, the magnetometer 266 and the strain gauge 260. Further, the device may include a GPS for determining the location of the user while the output data is processed. The geographical location of the user corresponding to the data can be stored inside the electronic device while synchronizing the device with the electronic device. This information can be used for evaluating location wise performance and stress of the user or group of users.

In one embodiment, user groups can be created on the cloud storage 400, and the aggregate data can be displayed on the electronic device (such as mobile, computer, tablet and the like) through the second processing module. This group can be of a company or any other group.

In yet another embodiment, GPS of the electronic device is used to identify geographical location of the user corresponding to the data stored. This data is used for evaluating location wise performance and mental wellness and physical wellness of the user.

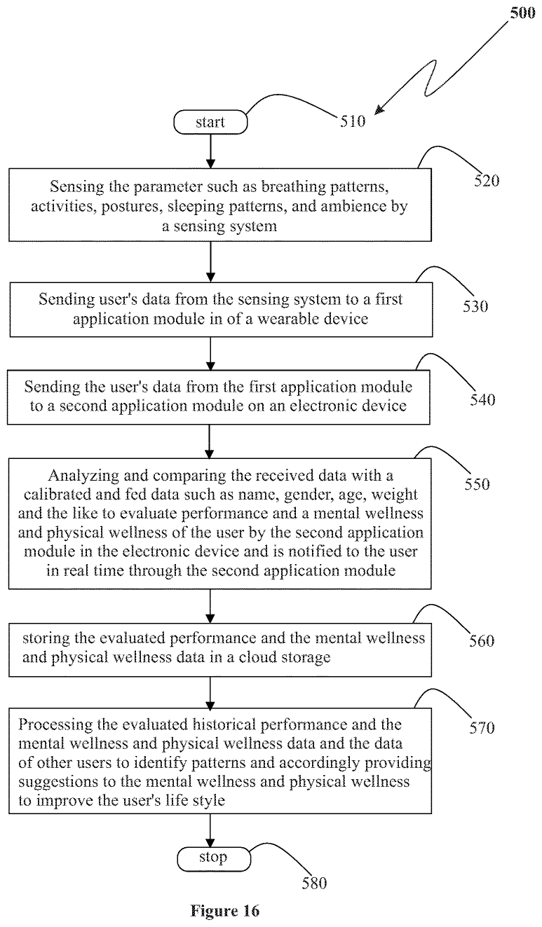

Referring now to FIG. 16, a method 500 for monitoring human performance in accordance with the present invention is illustrated. For the sake of brevity, the method 500 is explained in conjunction with the system 100.

The method 500 starts at step 510

At step 520, sensing the parameter such as breathing patterns, activities, postures, sleeping patterns, and ambience by a sensing system. In one embodiment, geographical location of the user corresponding to the data is stored and used for evaluating location wise performance and the mental wellness and physical wellness of the user.

At step 530, sending user's data from the sensing system to a first processing module in of a wearable device in real time.

At step 540, sending the user's data from the first processing module to a second processing module on an electronic device in real time.

Further, at step 550, analyzing and comparing the received data with a calibrated and fed data such as name, gender, age, weight and the like to evaluate performance and a mental wellness and physical wellness of the user by the second processing module in the electronic device and is notified to the user in real time through the second processing module.

At step 560, storing the evaluated performance and the mental wellness and physical wellness data in the cloud storage 400.

At step 570, processing the evaluated historical performance and the mental wellness and physical wellness data and the data of other users to identify patterns and accordingly providing suggestions to the mental wellness and physical wellness to improve the user's lifestyle.

The method ends at step 580.

Therefore, the present invention has an advantage of providing a wearable device 200 for monitoring human performance. The system 100 keeps real-time track of a mental and a physical health of a user. Specifically, the system 100 identifies a problem behind stress for the user in particular period of time. Further, the system 100 can send an alert to the user when their parameters are not matched with a fed or calibrated parameter. The system 100 helps the user to check breathing pattern, activity, posture, sleeping pattern and ambience. The device can process and provide data to an individual as well as to a corporate company for enhancement of work efficiency of the individual as well as group. The work efficiency of the user may be collected through the device and further process the collected data for assessment by human resource team of an organization to initiate required corrective actions. Further, the system 100 is capable to assist and monitor the user in real time. Also, the system 100 can analyze the user's personality as well as their compatibility with others.

The foregoing descriptions of specific embodiments of the present invention have been presented for purposes of illustration and description. They are not intended to be exhaustive or to limit the present invention to the precise forms disclosed, and obviously, many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the claims of the present invention.

I claim:

1. A system for monitoring human performance, the system comprising:
a wearable device having:
a sensing system configured to sense parameters such as breathing pattern, activity, posture, sleeping pattern, and ambience and sends data to a first processing unit having a first processing module;
an electronic device communicating wirelessly with the wearable device, the electronic device having:
a second processing unit configured to receive the data from the first processing module, the second processing unit process the data and sends data to a second processing module of the electronic device and compares with a calibrated and fed data to calculate a mental wellness and a physical wellness is notified to the user in real time through the second processing module, the data and the calculated state of the mental wellness and the physical wellness is stored on a cloud storage periodically, the breathing pattern, activity, sleeping pattern, posture, and ambience data also stored on the cloud storage, and
a third application module configured in the cloud storage, the third application module process the historical data of the mental wellness and the physical wellness of the user and the data of other users to identify patterns and accordingly providing suggestions to improve the mental wellness and the physical wellness.

2. The system as claimed in claim 1, wherein the sensing system comprises a light sensor, a noise sensor, a temperature sensor, an accelerometer, a gyroscope, a magnetometer, a Force Sensing Resistor sensor and a strain gauge.

3. The system as claimed in claim 1, wherein the breathing pattern is sensed by the FSR, the strain gauge, and the accelerometer.

4. The system as claimed in claim 1, wherein the wearable device is worn on a torso of a user.

5. The system as claimed in claim 1, wherein the second processing module analyses breathing patterns of the user to define a mental state.

6. The system as claimed in claim 1, wherein the wearable device is set in night mode before sleeping; during night mode the temperature, light and noise data is collected, and the data is sent to the first processing unit.

7. The system as claimed in claim 1, wherein for every user the posture is calibrated by reading standing straight posture and sitting straight posture.

8. The system as claimed in claim 1 and claim 2, wherein the user activity and sleeping pattern is sensed by the accelerometer, the magnetometer, and a gyroscope.

9. The system as claimed in claims 1 and 2, wherein the ambience is sensed by the light sensor, the noise sensor, and the temperature sensor.

10. The system as claimed in claims 1 and 2, wherein the posture is sensed by the accelerometer, the gyroscope, the magnetometer and the strain gauge.

11. The system as claimed in claim 1, wherein user groups can be created on the cloud, and the aggregate data can be displayed on the electronic device through the second processing module.

12. The system as claimed in claim 1, further using GPS of the electronic device geographical location of the user corresponding to the data is stored for evaluating location wise performance and mental wellness and physical wellness of the user.

13. A method for monitoring human performance, the method comprising steps of:
sensing the parameter such as breathing patterns, activities, postures, sleeping patterns, and ambience by a sensing system;
sending user's data from the sensing system to a first processing module in of a wearable device;
sending the user's data from the first processing module to a second processing module on an electronic device;
analyzing and comparing the received data with a calibrated and fed data such as name, gender, age, weight and the like, to evaluate performance and a mental wellness and physical wellness of the user by the second processing module in the electronic device and is notified to the user in real time through the second processing module;
storing the evaluated performance and the mental wellness and physical wellness data in a cloud storage;
processing the evaluated historical performance and the mental wellness and physical wellness data and the data of other users to identify patterns and accordingly providing suggestions to the mental wellness and physical wellness to improve the user's lifestyle.

14. The method as claimed in claim 13, further storing geographical location of the user corresponding to the data for evaluating location wise performance and the mental wellness and physical wellness of the user.
